(19) United States
(12) Patent Application Publication
(10) Pub. No.: US 2017/0253844 A1
(43) Pub. Date: Sep. 7, 2017

(54) BOWL SHAPED MICROWELL

(71) Applicant: Corning Incorporated, Corning, NY (US)

(72) Inventors: Ye Fang, Painted Post, NY (US); Vasiliy Nikolaevich Goral, Painted Post, NY (US)

(21) Appl. No.: 15/449,222

(22) Filed: Mar. 3, 2017

Related U.S. Application Data
(60) Provisional application No. 62/303,606, filed on Mar. 4, 2016.

(51) Int. Cl. C12M 1/32; C12N 5/00; C12M 1/04

(52) U.S. Cl. CPC C12M 23/12; C12M 23/24; C12N 5/0062

(57) ABSTRACT

A cell culture apparatus has a structure defining a cell culture microwell. The cell culture microwell defines a top aperture and an inner surface and an axis extending through the top aperture. The top aperture defines a top diametric dimension measured perpendicular to the axis and the inner surface defines a first diametric dimension measured perpendicular to the axis at a widest portion of the microwell and a second diametric dimension measured perpendicular to the axis. The second diametric dimension is at a location along the axis farther from the top aperture than a location along the axis of the first diametric dimension. The top diametric dimension is less than the first diametric dimension, and the second diametric dimension is less than the first diametric dimension.

This application claims the benefit of priority under 35 U.S.C. §119 of US Application Ser. Nos. 62/130,804 filed on Mar. 10 2015 and 62/303,606 filed on Mar. 4, 2016, the contents of each are relied upon and incorporated herein by reference in their entirety.

### FIELD

The present disclosure relates to devices, systems and methods for culturing cells.

### TECHNICAL BACKGROUND

Cells cultured in three dimensions, such as spheroids, can exhibit more in vivo-like functionality than their counterparts cultured in two dimensions as monolayers.

A number of techniques for spheroid formation have been reported. For example, colonization of cells on scaffold surfaces that favor cell-cell interaction rather than cell-substratum interaction has been used to culture spheroids. Hanging drop methods, rotational culture and agitation culture have also been employed to culture cells as spheroids. Another method for spheroid formation involves culturing cells in microwells having low attachments surfaces. However, culturing cells in conditions that provide an environment conducive to spheroid formation continues to be challenging. Providing sufficient nutrients and allowing for media changes, controlling the size of spheroids, capturing and handling spheroids, and manipulating spheroids for use in assays, for example, remain challenging tasks. There is a need for improved cell culture devices that support the formation, culture, manipulation and use of spheroids.

### BRIEF SUMMARY

The present disclosure relates to, among other things, cell culture devices, systems and methods for culturing cells in three-dimensions. In some embodiments, the devices, systems and methods disclosed herein allow cells to be cultured as spheroids of consistent size and allow the use of routine liquid manipulations without cells being lost.

In accordance with various embodiments, the present disclosure describes a cell culture apparatus having a structure defining a cell culture microwell. The cell culture microwell defines a top aperture, an inner surface, and an axis extending through the top aperture. The top aperture defines a top diametric dimension measured perpendicular to the axis and the inner surface defines a first diametric dimension measured perpendicular to the axis at a widest portion of the cell culture microwell and a second diametric dimension measured perpendicular to the axis. The second diametric dimension is at a location along the axis farther from the top aperture than a location along the axis of the first diametric dimension. The top diametric dimension is less than the first diametric dimension, and the second diametric dimension is less than the first diametric dimension.

In accordance with various embodiments, the present disclosure describes a cell culture apparatus having a structure defining a well channel. The well channel defines a top aperture, a bottom aperture and a sidewall surface between the top and bottom aperture. The top aperture defines a top diametric dimension and the bottom aperture defines a bottom diametric dimension smaller than the top diametric dimension. The bottom diametric dimension is in a range from 25 micrometers to 4,000 micrometers. The well channel is configured to hang a drop of cell culture fluid below the bottom aperture to form a cell culture microwell.

Additional features and advantages of the subject matter of the present disclosure will be set forth in the detailed description which follows, and in part will be readily apparent to those skilled in the art from that description or recognized by practicing the subject matter of the present disclosure as described herein, including the detailed description which follows, the claims, as well as the appended drawings.

It is to be understood that both the foregoing general description and the following detailed description present embodiments of the subject matter of the present disclosure, and are intended to provide an overview or framework for understanding the nature and character of the subject matter of the present disclosure as it is claimed. The accompanying drawings are included to provide a further understanding of the subject matter of the present disclosure, and are incorporated into and constitute a part of this specification. The drawings illustrate various embodiments of the subject matter of the present disclosure and together with the description serve to explain the principles and operations of the subject matter of the present disclosure. Additionally, the drawings and descriptions are meant to be merely illustrative, and are not intended to limit the scope of the claims in any manner.

### BRIEF DESCRIPTION OF THE DRAWINGS

The following detailed description of specific embodiments of the present disclosure can be best understood when read in conjunction with the following drawings, in which:

FIG. 1 is a cross-sectional view of an embodiment of a cell culture apparatus having a plurality of microwells.

FIG. 2A is a cross-sectional view of an embodiment of a cell culture microwell.

FIG. 2B is a cross-sectional view of an embodiment of a cell culture microwell.

FIG. 3A is a cross-sectional view of an embodiment of a structure that includes a cell culture microwell.

FIG. 3B is a cross-sectional view of an embodiment of a structure that includes a cell culture microwell.

FIG. 4A is a cross-sectional view of an embodiment of a cell culture apparatus having a plurality of microwells.

FIG. 4B is a cross-sectional view of an embodiment of a cell culture apparatus having a plurality of microwells.

FIG. 5 is a cross-sectional view of an embodiment of a well channel with cell culture medium forming a microwell.

FIG. 6A is a cross-sectional view of an embodiment of a well channel and reservoir.

FIG. 6B is a cross-sectional view of an embodiment of a well channel and reservoir.

FIG. 7 is a cross-sectional view of an embodiment of a plurality of well channels and reservoirs.

FIG. 8 is a perspective view of an embodiment of a cell culture apparatus having a plurality of wells.

FIG. 9 is a schematic perspective view of an embodiment of a cell culture apparatus having a plurality of wells.

FIG. 10A is a schematic perspective view of a blow molding process of an embodiment.

FIG. 10B is a schematic cross-sectional view of a blow molding process of an embodiment.

The schematic drawings are not necessarily to scale.

### DETAILED DESCRIPTION

Reference will now be made in greater detail to various embodiments of the subject matter of the present disclosure, some embodiments of which are illustrated in the accompanying drawings. Like numbers used in the figures refer to like components, steps and the like. However, it will be understood that the use of a number to refer to a component in a given figure is not intended to limit the component in another figure labeled with the same number. In addition, the use of different numbers to refer to components in different figures is not intended to indicate that the different numbered components cannot be the same or similar to other numbered components.

The present disclosure describes, among other things, cell culture devices having a structure defining a cell culture microwell (or microwell) and optionally a well channel. The cell culture microwell defines a cell culturing volume that helps control the growth of cells (e.g., in the form of spheroids, as further described herein).

In some embodiments, the microwells may be configured such that cells cultured in the microwells form spheroids. For example, the microwells can be non-adherent to cells to cause the cells in the microwells to associate with each other and form spheroid clusters. In some embodiments, the microwells may be coated with a cell non-adherent material (i.e., an ultra-low binding material) to make the microwells non-adherent to cells. Examples of non-adherent materials include perfluorinated polymers, olefins, or like polymers or mixtures thereof. Other examples may include agarose, non-ionic hydrogels such as polyacrylamides, or like materials or mixtures thereof. The non-adherent materials can include polyethylene glycol (PEG) silane when the microwell substrate is glass. The combination of, for example, non-adherent microwells, microwell geometry, and gravity may induce cells cultured in the microwells to self-assemble into spheroids. For certain cell types, spheroids can maintain differentiated cell function indicative of a more in vivo like response relative to cells grown in a monolayer.

In some embodiments, one or more of the microwells are configured to grow a single spheroid of a defined size in each microwell. Spheroids may expand to size limits imposed by the geometry of the microwells in which they are cultured. For example, each microwell may allow the spheroid to grow to a certain diameter. In other words, the geometrical dimensions of the microwell may constrain the spheroid growth such that the spheroid diameter may reach a maximum value and stay at that maximum value as defined by the geometry of the microwell. The production of consistently sized spheroids may lead to tissue-like, non-expanding spheroids that may be ideal for improving reproducibility of assay results. The production of consistent spheroids may be the result of consistently shaped and dimensioned volumes (e.g., a microwell or cell culture volume) defined by the inner surface of the microwell. For example, the microwell may, in some embodiments, have a generally spherical shape with a diametric dimension in a range from about 100 micrometers to about 6000 micrometers at a widest portion of the generally spherical shape.

In some embodiments, a cell culture apparatus can include a well channel that defines a bottom aperture through which a drop of cell culture medium may hang and form a virtual microwell. In such embodiments, the cell culture apparatus; e.g., the well channel and bottom aperture, and the cell culture medium cooperate to form the microwell. The cell culture medium stays in place hanging from the aperture due to surface tension. The size of the aperture, the material forming the well channel, the composition of the cell culture medium, and the weight of cell culture medium disposed above the hanging drop factor into the shape and dimensions of the hanging drop and factor into the ability of the cell culture medium to maintain a surface tension that allows the drop to hang from the aperture. The geometry of the hanging drop then constrains the spheroid growth such that the spheroid diameter may reach a maximum value and stay at that maximum value. The production of consistently sized spheroids may lead to tissue-like, non-expanding spheroids that may be ideal for improving reproducibility of assay results. In some embodiments, the bottom aperture of the well channel may have a diametric dimension in a range from about 50 micrometers to 2000 micrometers.

Nearly any type of cell culture apparatus having a well used to culture cells may be designed to employ a microwell as described herein. For example, the microwell can form the entire volume of the well or, in some embodiments, can form a cell culturing sub-volume positioned within the well. In some embodiments, the cell culture devices with which the microwell design may be implemented may be a multi-well plate, for example, a 96-well multiwell plate, a 384-well multiwell plate, a 1536-well multiwell plate, or the like, as discussed herein.

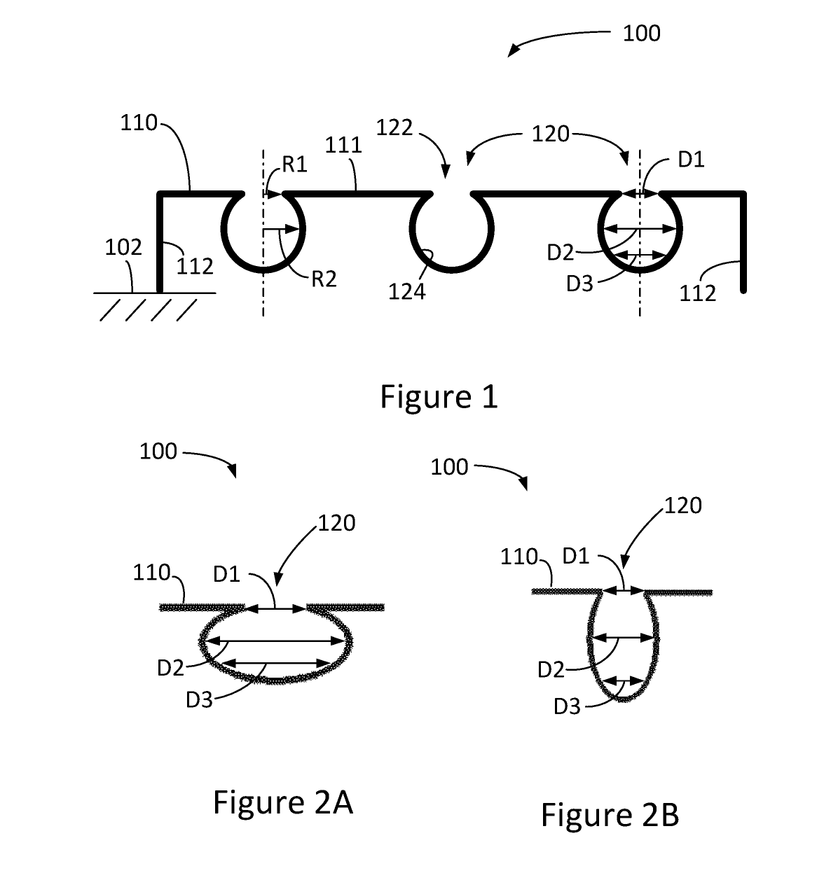

Referring now to FIG. 1, a side cross-sectional view of an embodiment of a cell culture apparatus 100 including a structure 110 defining a plurality of microwells 120 is shown. Each of the plurality of microwells 120 may define a top aperture 122 and an inner surface 124. The top aperture 122 of each of the plurality of microwells 120 may be used as an opening through which to seed cells into each of the plurality of microwells 120. The top aperture 122 may have a variety of different shapes and sizes. For example, the top aperture 122 may be defined by a shape that is circular, oval, square, rectangular, hexagonal, quadrilateral, etc. The top aperture 122 may also be defined by a diametric dimension D1 (e.g., diameter, width, etc., depending on shape). The top aperture may also be defined by a radius R1 (e.g., a length from an edge of the top aperture 122 to an axis of measurement extending normal to and through the center of the top aperture 122). The distance across the top aperture 122 at a widest portion may be defined by the diametric dimension D1 or twice the radius R1. Accordingly, the diametric dimension D1 may be double the radius R1.

The radius R1 of the top aperture 122 may be about, e.g., greater than or equal to 20 micrometers, greater than or equal to 40 micrometers, greater than or equal to 100 micrometers, greater than or equal to 250 micrometers, greater than or equal to 500 micrometers, greater than or equal to 650 micrometers, etc. or, less than or equal to 3000 micrometers, less than or equal to 2000 micrometers, less than or equal to 1500 micrometers, less than or equal to 1000 micrometers, less than or equal to 750 micrometers, less than or equal to 400 micrometers, etc. or any range within the aforementioned values. Accordingly, the diametric dimension D1 of the top aperture 122 may be about, e.g., greater than or equal to 40 micrometers, greater than or equal to 80 micrometers, greater than or equal to 200 micrometers, greater than or equal to 500 micrometers, greater than or equal to 1000 micrometers, greater than or equal to 1300 micrometers, etc. or, less than or equal to 6000 micrometers, less than or equal to 4000 micrometers, less than or equal to 3000 micrometers, less than or equal to 2000 micrometers, less than or equal to 1500 micrometers, less than or equal to 800 micrometers, etc. or any range within the aforementioned values.

The inner surface 124 of each of the plurality of microwells 120 may be conducive to allowing cells to be cultured thereon or there-above. The inner surface 124 may have a variety of different bowl-like shapes and sizes. For example, the inner surface 124 may be rounded, hemispherical, semispherical, etc. As shown in FIG. 1, the inner surface 124 of the microwell 120 has a semispherical shape and the depicted cross-section of the microwell 120 is circular. The inner surface 124 may also be defined by a first diametric dimension D2 (e.g., diameter, width, etc.) or a radius R2 (e.g., a length from the inner surface 124 to an axis of measurement extending normal to and through the center of the top aperture 122). The first diametric dimension D2 or radius R2 is measured at the widest portion of the inner surface 124 of the microwell 120. As shown in FIG. 1, the first diametric dimension is measured perpendicular to an axis extending through the top aperture. The first diametric dimension D2 may be double the radius R2. The inner surface 124 may also define a second diametric dimension D3 that is measured perpendicular to the axis and at a location along the axis farther from the top aperture 122 than a location along the axis of the first diametric dimension D2.

The radius R2 of the widest portion of the inner surface 124 of the microwell 120 may be about, e.g., greater than or equal to 25 micrometers, greater than or equal to 50 micrometers, greater than or equal to 100 micrometers, greater than or equal to 250 micrometers, greater than or equal to 500 micrometers, greater than or equal to 750 micrometers, etc. or, less than or equal to 4000 micrometers, less than or equal to 3000 micrometers, less than or equal to 2000 micrometers, less than or equal to 1000 micrometers, less than or equal to 650 micrometers, less than or equal to 450 micrometers, etc. or any range within the aforementioned values. Accordingly, the first diametric dimension D2 of the widest portion of the inner surface 124 of the microwell 120 may be about, e.g., greater than or equal to 50 micrometers, greater than or equal to 100 micrometers, greater than or equal to 200 micrometers, greater than or equal to 500 micrometers, greater than or equal to 1000 micrometers, greater than or equal to 1500 micrometers, etc. or, less than or equal to 8000 micrometers, less than or equal to 6000 micrometers, less than or equal to 4000 micrometers, less than or equal to 2000 micrometers, less than or equal to 1300 micrometers, less than or equal to 900 micrometers, etc. or any range within the aforementioned values.

As shown in FIG. 1, the diametric dimension D1 of top aperture 122 is less than first the diametric dimension D2 of the inner surface 124 at the widest portion of the microwell 120. Alternatively, the first diametric dimension D2 of the inner surface 124 at the widest portion of the microwell 120 may be described as greater than the diametric dimension D1 of the top aperture 122. Such a configuration allows for individual cells that are seeded in the culture apparatus in culture medium to enter the microwell 120 through the top aperture 122. As the cells are cultured, form spheroids and grow, the diametric dimensions of the spheroid (defined by dimensions of the microwell 120) may be too large to be readily removed through the top aperture 122. As such, routine or automated liquid handling techniques may be used to remove or replace culture medium without loss of cells through, for example, aspiration or dumping of cell culture medium. Also as shown in FIG. 1, the second diametric dimension D3 is less than the first diametric dimension D2. In the depicted embodiment, D3 can approach zero depending on the location along the axis that D3 is measured (e.g., if D3 is measured at or near the nadir of the inner surface of the microwell).

In embodiments, a ratio of the diametric dimension D1 of the top aperture to the first diametric dimension D2 (i.e., D1/D2) is 0.1, 0.2, 0.3, 0.4, 0.5, 0.6, 0.7, 0.8 or 0.9, including ranges between any of the foregoing. In embodiments, a ratio of the second diametric dimension D3 to the first diametric dimension D2 (i.e., D3/D2) is 0.1, 0.2, 0.3, 0.4, 0.5, 0.6, 0.7, 0.8 or 0.9, including ranges between any of the foregoing.

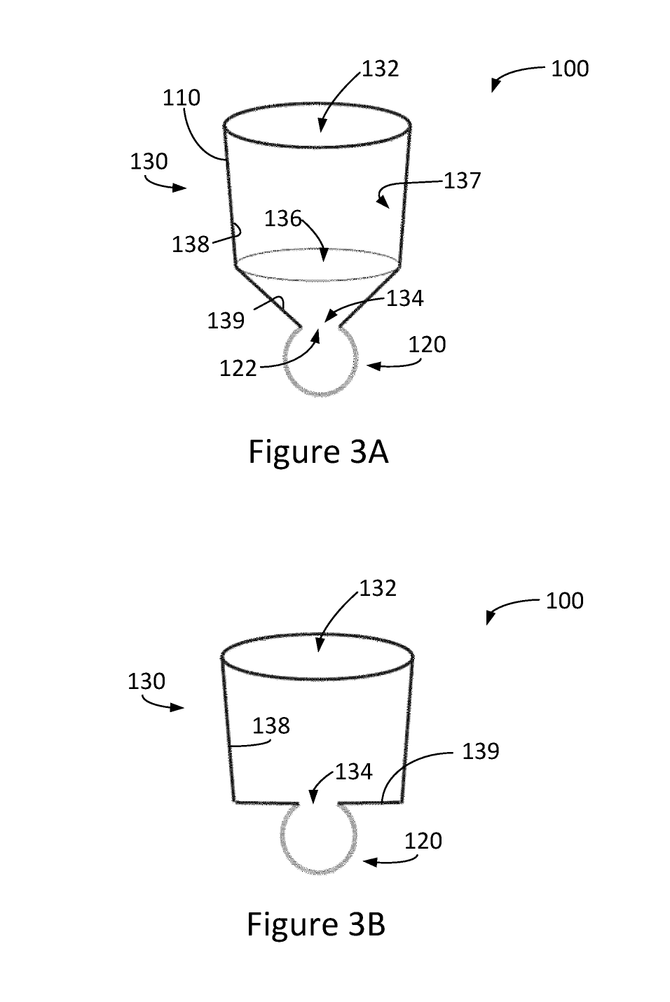

In the embodiment depicted in FIG. 1, the structure 110 also defines a flat surface 111 between the plurality of microwells 120 that is on a plane parallel with a plane defined by the top apertures 122 of the microwells 120. In other embodiments, for example as shown in FIGS. 2A and 2B, the structure 110 defines one microwell 120 that is independent from any other microwell 120. As will be discussed further herein, the structure 110 defining a single microwell 120 or a plurality of microwells 120 may be configured to be placed within other wells as an insert.

In the embodiments depicted in FIG. 1 or other embodiments depicted and described herein, the inner surface 124 of the microwell 120 may be gas permeable to help provide oxygen to the cells or spheroids cultured within the microwell 120. In some embodiments, the structure 110 that defines the inner surface 124 may be a gas permeable substrate or gas permeable film. The gas permeability of the inner surface 124 to the exterior environment will depend in part on the material of the inner surface 124 and the thickness of the inner surface 124. For example, the gas permeability of the microwell may be as described in U.S. provisional patent application No. 62/072,088, filed on 29 Oct. 2014, and entitled “GAS PERMEABLE CULTURE FLASK,” which provisional patent application is hereby incorporated herein by reference in its entirety to the extent that it does not conflict with the present disclosure.

The structure 110 of the cell culture apparatus 100 may also include one or more supports 112. For example, the support 112 may be configured to position the microwell 120 above a surface 102 (e.g., ground, table, etc.) to prevent damage to the microwell 120, to allow airflow below the microwell 120, or the like. In some embodiments, the structure 110 may include a plurality of supports 112 to balance the microwells 120 and allocate the weight of the structure 110 (e.g., a support 112 may be located in each of four corners of the structure 110). In some embodiments, the microwell 120 or plurality of microwells 120 may be able to support itself or themselves without the assistance of one or more separate supports 112.

In the embodiments depicted in FIGS. 2A and 2B, the microwells 120 have an oblong semispherical shape with an oval shaped cross-section. Similar to the microwells depicted in FIG. 1, each of the microwells 120 depicted in FIGS. 2A and 2B have a first diametric dimension D2 measured perpendicular to an axis extending through the top aperture 122 at a widest portion of the inner surface of the microwell 120 that is greater than a diametric dimension D1 of the top aperture of the microwell 120. Accordingly, individual cells that are seeded in the culture apparatus in culture medium can enter the microwell 120 through the top aperture 122. As the cells are cultured, form spheroids and grow, the diametric dimensions of the spheroid (e.g., defined by dimensions of the microwell 120) may be too large to be readily removed through the top aperture 122. As such, routine or automated liquid handling techniques may be used to remove or replace culture medium without loss of cells through, for example, aspiration or dumping of cell culture medium. Also, each of the microwells 120 depicted in FIGS. 2A and 2B defines a second diametric dimension D3 that is less than the first diametric dimension D2, similar to FIG. 1.

FIG. 2A illustrates a microwell 120 having a width greater than its depth. FIG. 2B illustrates a microwell 120 having a depth greater than its width. Of course, cell culture devices as described herein can have any suitable shape or dimension.

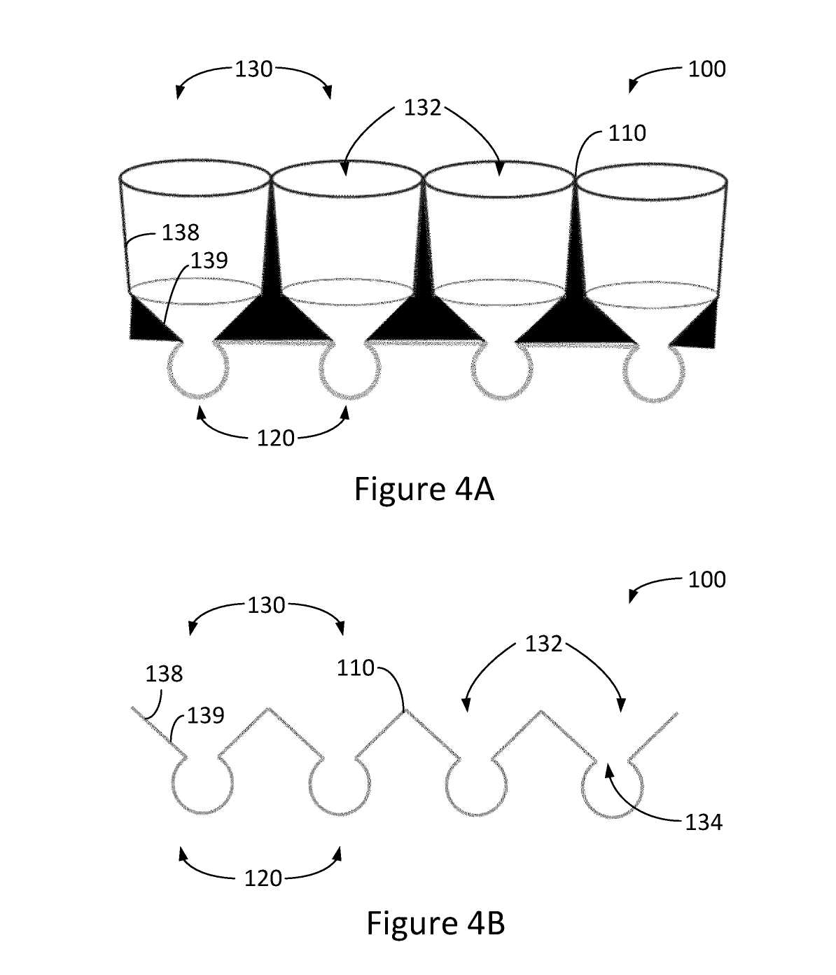

Referring now to FIG. 3A, a cross-sectional view of one embodiment of a microwell 120 of cell culture apparatus 100 is illustrated. In the depicted embodiment, the structure 110 of the cell culture apparatus 100 defines a well channel 130 above the microwell 120 and in fluid communication with the top aperture 122 of the microwell 120. The well channel 130 defines a top aperture 132, a bottom aperture 134, a sidewall surface 137 extending from the top aperture 132 to the bottom aperture 134, and a middle aperture 136 between the top aperture 132 and the bottom aperture 134. In some embodiments, the bottom aperture 134 may be the same as the top aperture 122 of the microwell 122. In other words, the structure 110 may transform from the well channel 130 into the microwell 120 at the bottom aperture 134 of the well channel 130 (also described as the top aperture 122 of the microwell 120). The top aperture 132 of the well channel 130 may have any of a variety of different shapes and sizes. For example, the top aperture 132 of the well channel 130 may be defined by a shape that is circular, oval, square, rectangular, hexagonal, quadrilateral, etc.

The top aperture 132 and bottom aperture 134 of the well channel 130 may each be defined by a diametric dimension. The diametric dimensions of the top aperture 132 and bottom aperture 134 can be measured at their respective widest portions. In some embodiments, the diametric dimension of the top aperture 132 of the well channel 130 can be greater than or equal to the diametric dimension of the bottom aperture 134 of the well channel 130. In other words, the diametric dimension of the bottom aperture 134 of the well channel 130 may be less than the diametric dimension of the top aperture 132 of the well channel 130. The middle aperture 136 of the well channel 130 may also be defined by a diametric dimension that may be measured in a plane of the middle aperture 136 (e.g., a plane that is parallel with the top or bottom apertures 132, 137) at a point along the sidewall surface 137. In some embodiments, the diametric dimension of the top aperture 132 of the well channel 130 is greater than or equal to the diametric dimension of the middle aperture 136 of the well channel 130 and the diametric dimension of the middle aperture 136 of the well channel 130 is greater than or equal to the diametric dimension of the bottom aperture 134 of the well channel 130.

The shape of the sidewall surface 137 may be configured to promote the progress of cells in the well channel 130 to move towards the microwell 120 due to gravity. In some embodiments, the sidewall is tapered such that the diametric dimension of the sidewall surface decreases along at least a portion of the sidewall moving towards the bottom. For example, the sidewall surface 137 of the well channel 130 may be defined by a constant angle from the top aperture 132 to the bottom aperture 134, from the top aperture 132 to the middle aperture 136, or from the middle aperture 136 to the bottom aperture 134. In other embodiments, the sidewall surface 137 may be defined by a curved surface (e.g., a parabolic shape, etc.) between any of the three different apertures 132, 134, 136 as described above.

The sidewall surface 137 of the well channel 130 may be further described as an upper sidewall surface 138 and a lower sidewall surface 139. The upper sidewall surface 138 of the well channel 130 may be defined between the top aperture 132 and the middle aperture 136 of the well channel 136. The lower sidewall surface 139 of the well channel 130 may be defined between the middle aperture 136 and the bottom aperture 134 of the well channel. The upper and lower sidewall surfaces 138, 139 may be defined by the same or different linear or curved surfaces (e.g., the upper sidewall surface 138 may have one constant angle and the lower sidewall surface 139 may have another constant angle, the upper sidewall surface 138 may have a constant angle and the lower sidewall surface 139 may have a curved surface, etc.). As shown in FIG. 3B, the lower sidewall surface 139 may define a plane that is parallel with a plane defined by the top aperture 132 (or bottom aperture 134) of the well channel 130. In other words, the lower sidewall surface 139 may be in a same plane as the bottom aperture 134 of the well channel 130.

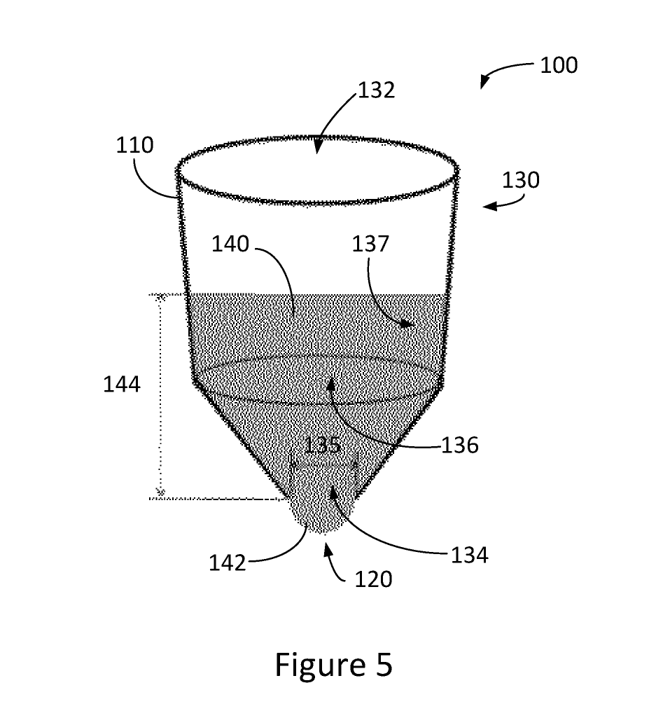

In some embodiments, the cell culture apparatus 100 may include a plurality of microwells 120 as shown in FIGS. 4A and 4B. Each of the plurality of microwells 120 has an independent well channel 130 leading to the microwell 120. The structure 110 of the cell culture apparatus 100 depicted in FIGS. 4A and 4B defines both the plurality of microwells 120 and the plurality of well channels 130. However, it will be understood that embodiments where separate structures define the microwells and the well channels are contemplated herein.

In embodiments depicted in FIGS. 4A and 4B, the structure 110 connecting the plurality of microwells 120 allows for the plurality of microwells 120 to be moved and placed in unison. FIG. 4A illustrates a well channel 130 including two different angles on the upper and lower sidewall surfaces 138, 139. FIG. 4B illustrates a well channel 130 including only one angle across both the upper and lower sidewall surfaces 138, 139, creating one constant angle of the sidewall surface from the top aperture 132 to the bottom aperture 134 of the well channel 130. The top apertures 132 of the well channels 130 are proximate or touching each of the top apertures 132 of the adjacent well channels 130 in the embodiments depicted in FIGS. 4A and 4B. However, it will be understood that embodiments where the top apertures of well channels are spaced apart are contemplated herein.

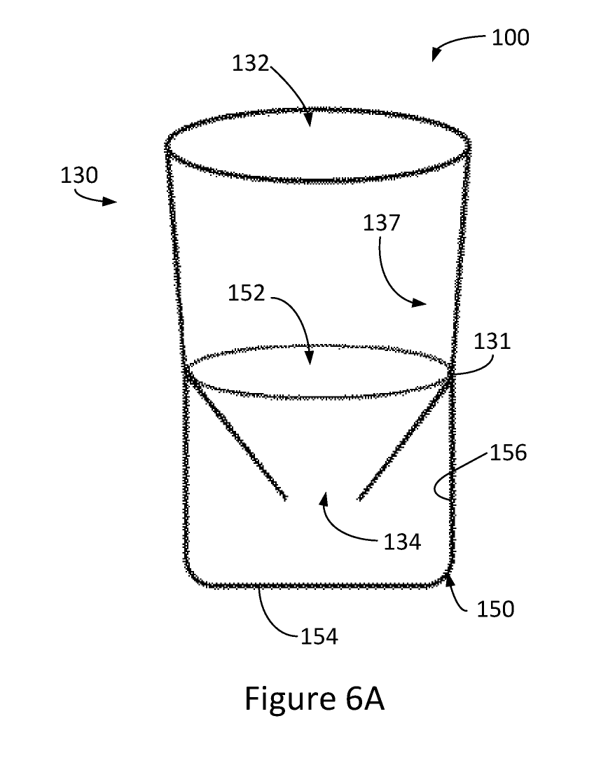

Referring now to FIG. 5, a side cross-sectional view of an embodiment of a cell culture apparatus 100 including a structure 110 defining a well channel 130 is shown. The well channel 130 defines a top aperture 132, a bottom aperture 134, and a sidewall surface 137 extending from the top aperture 132 to the bottom aperture 134. The top aperture 132 of the well channel 130 may be used as an opening through which to seed cells and introduce cell culture medium 140 into the cell culture apparatus 100. The top aperture 132 may have a variety of different shapes and sizes. For example, the top aperture 132 may be defined by a shape that is circular, oval, square, rectangular, hexagonal, quadrilateral, etc. The top aperture 132 and bottom aperture 134 may be defined by a diametric dimension (e.g., diameter, width, etc., depending on shape). The diametric dimension 135 of the bottom aperture 134 may be less than the diametric dimension of the top aperture 132. The diametric dimensions of the top and bottom apertures 132, 134 may be defined as a distance across the widest portion of the corresponding aperture. The diametric dimension 135 of the bottom aperture 134 of the well channel 130 may be about, e.g., greater than or equal to 25 micrometers, greater than or equal to 50 micrometers, greater than or equal to 100 micrometers, greater than or equal to 250 micrometers, greater than or equal to 500 micrometers, greater than or equal to 650 micrometers, etc. or, less than or equal to 3000 micrometers, less than or equal to 2000 micrometers, less than or equal to 1500 micrometers, less than or equal to 1000 micrometers, less than or equal to 750 micrometers, less than or equal to 400 micrometers, etc. or any range within the aforementioned values.

The well channel 130 may also include features similar to those described above with reference to FIGS. 2A and 2B. For example, the well channel 130 may define a middle aperture 136 between the top and bottom apertures 132, 134 along the sidewall surface 137. The sidewall surface 137 of the well channel 130 may also have a variety of shapes, as discussed previously.

Still with reference to FIG. 5, the bottom aperture 134 of the well channel 130 is configured to hang a drop 142 of cell culture fluid 140 (or medium) below the bottom aperture 134. The hanging drop 142 forms a microwell 120 of cell culture medium 140. Such a virtual microwell 120 provides similar characteristics as described above regarding the microwell 120 of FIGS. 1-4. For example, cell-cell interaction and spheroid growth are favored.

Surface tension maintains the drop 142 in contact with the bottom aperture 134 of the well channel 130 due to characteristics of the fluid, the size of the bottom aperture 134, material forming the bottom aperture 134, and the amount of fluid present in the well channel 130. The combination of these factors provides a hanging drop 142 from the bottom aperture 134 of the well channel 130. With regard to the amount of fluid present in the well channel 130, the cell culture medium 140 is filled up to a certain height 144 within the well channel 130, for a given diametric dimension 135 of the bottom aperture 134 of the well channel 130. A differing height 144 of cell culture medium 140 provides a differing weight pressing down on the hanging drop 142. Therefore, an appropriate balance between characteristics of the cell culture medium 140, the diametric dimension 135 of the bottom aperture 134 of the well channel 130, and the height 144 of cell culture fluid 140 provides a hanging drop 142 of cell culture medium 140 or virtual microwell 120 that maintains its position relative to the bottom aperture 134 of the well channel 130 provided that the cell culture apparatus 100 remains stationary in a cell culture position.

The hanging drop 142 or virtual microwell 120 of cell culture medium 140 has a generally semispherical shape. Cells that are seeded into the well channel 130 then aggregate in the hanging drop 142 or virtual microwell 120 to form a spheroid. The cells are restricted by the bounds of the cell culture medium 140 in the hanging drop 142 or virtual microwell 120. The hanging drop 142 is formed in such a way that the cells seeded into the well channel 130 do not disturb the structure of the hanging drop 142 and do not, at least initially, cause the hanging drop 142 to fall and separate from the bottom aperture 134 of the well channel 130.

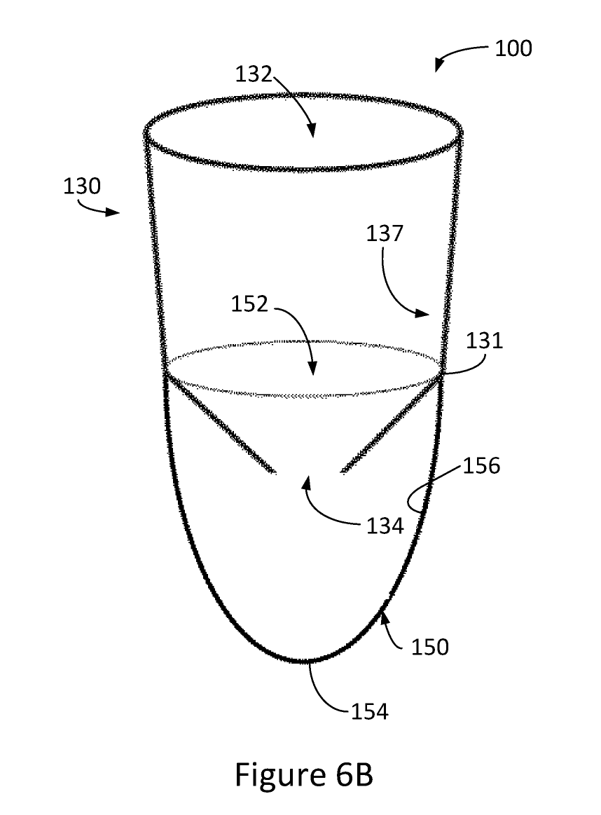

FIGS. 6A and 6B illustrate the cell culture apparatus 100 of FIG. 5 further including a reservoir 150. The reservoir 150 defines a top aperture 152, a bottom surface 154, and a sidewall surface 156 extending from the top aperture 152 to the bottom surface 154. The bottom surface 154 of the reservoir 150 may be located below the bottom aperture 134 of the well channel 130. A seal may or may not be formed between the sidewall surface 156 of the reservoir 150 and an exterior surface 131 of the well channel 130 at the top aperture 152 of the reservoir 150, with the exterior surface 131 of the well channel 130 being opposite the sidewall surface 137 of the well channel 130. In other words, the reservoir 150 can be closed off from the environment except for through the bottom aperture 134 of the well channel 130. If the sidewall 156 is relatively impermeable to air and if a seal is formed between the sidewall surface 156 and the exterior surface 131 of the well channel 130, air within the reservoir may be essentially trapped during use due to limited permeability through the cell culture medium (not depicted in FIGS. 6A and 6b) within the well channel 130. The relative inability for air to escape the reservoir 150 provides an extra force to help maintain the hanging drop in position in the bottom aperture 134 of the well channel 130. Additionally, a closed off reservoir 150 can help prevent external elements (e.g., air movement, etc.) that may disrupt the hanging drop. In other words, the hanging drop can be more susceptible to falling from the bottom aperture 134 of the well channel 130 if there is no reservoir 150.

Although, in some embodiments, the reservoir 150 may include a venting aperture (not shown) or can include air permeable sidewalls 156 to allow passage of air or fluid into and out of the reservoir 150. While such embodiments may not benefit from the force of trapped air to maintain the hanging drop, such embodiments benefit from enhanced gas exchange between cells cultured in the hanging drop and the external environment.

The reservoir 150 may also be used to contain the spheroid after culturing. For example, after the spheroid is grown in the virtual microwell or hanging drop of cell culture medium, the hanging drop may be disrupted to cause the spheroid to fall into the reservoir 150. For certain assays or microscopic inspection, it can be beneficial for the spheroids to be adjacent the bottom surface 154 of the reservoir 150.

In the embodiment shown in FIG. 6A, the bottom surface 154 of the reservoir 150 defines a flat planar surface that is parallel with a plane defined by the bottom aperture 134 of the well channel 130. A flat surface of the reservoir 150 may be optically advantageous for microscopy of spheroids. As shown in FIG. 6B, the bottom surface 154 of the reservoir 150 may define a parabolic shape. Such a parabolic shape may be advantageous for assaying spheroids contained within a confined volume defined by the parabolic shape.

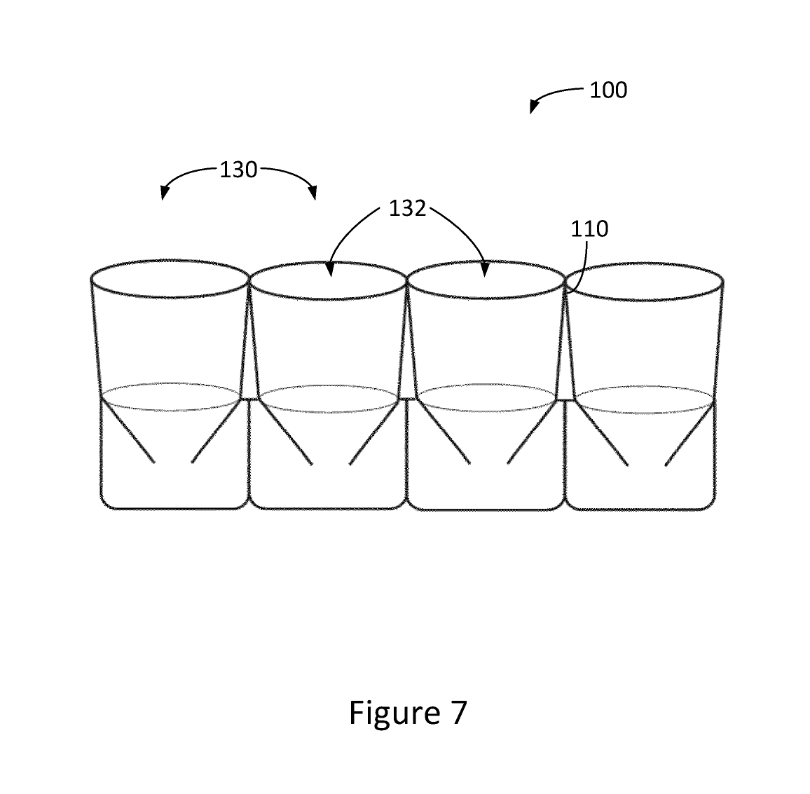

In some embodiments, the cell culture apparatus 100 can include a plurality of well channels 130 as shown in, for example, FIG. 7. The structure 110 connecting the plurality of well channels 130 allows for the plurality of well channels 130 to be moved and placed in unison. The top apertures 132 of the well channels 130 are proximate or touching each of the top apertures 132 of the adjacent well channels 130. However, it will be understood that embodiments where the top apertures of well channels are spaced apart are contemplated herein.

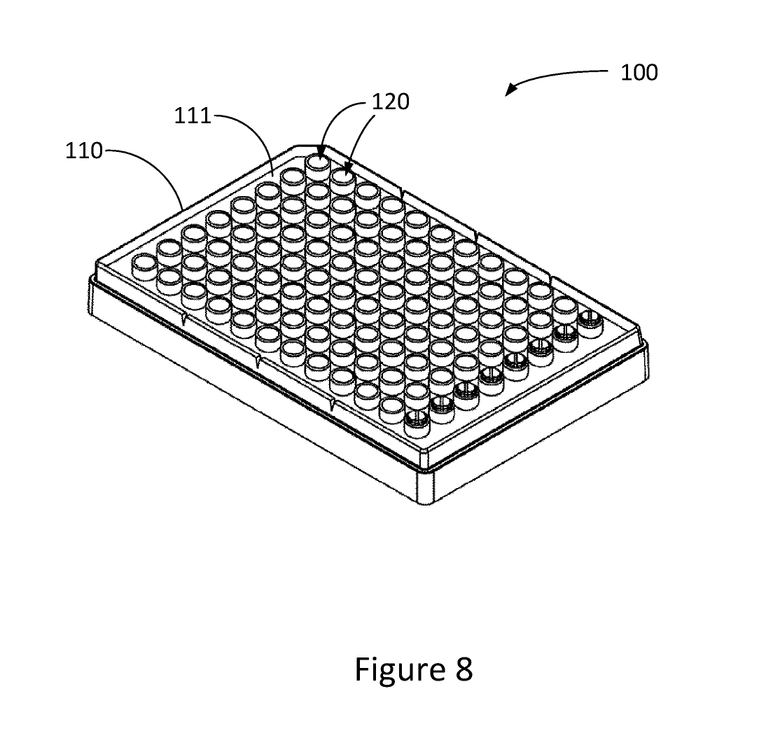

In the embodiment depicted in FIG. 8, the cell culture apparatus 100 is a 96-well multiwell plate. However, as discussed above, a cell culture apparatus 100 as described herein can have any suitable number of wells. In the depicted embodiment, at least a portion of each of the plurality of microwells 120 drops below a major surface 111 of the structure 110 of the cell culture apparatus 100. In some embodiments, the multiwell plate may be constructed to contain a microwell 120 in each of the wells. In other embodiments, the structure defining the microwells 120 may be configured to be placed inside of an existing multiwell plate. In other words, the structure can be an insert for insertion into one or more wells of the multiwell plate. Further, the cell culture apparatus 100 may be configured such that multiple microwells are inserted into each well of the multiwell plate. The structure may define only one microwell, therefore allowing either one or a plurality of structures each defining one microwell to be placed independently within a respective well of the multiwell plate. The structure may also define a plurality of microwells that may each be placed within respective wells of a multiwell plate simultaneously.

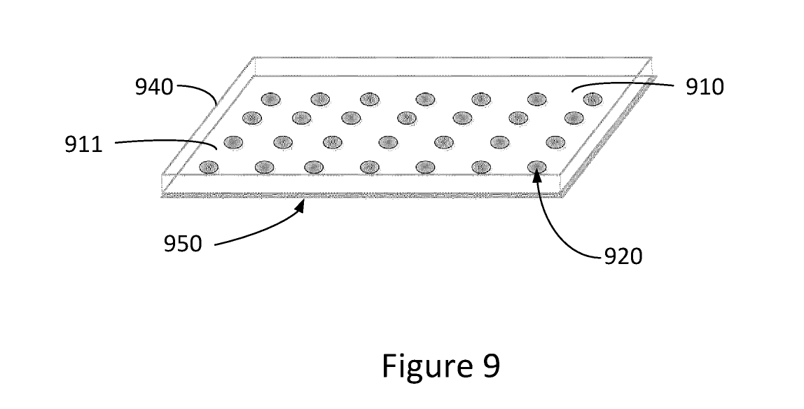

In some embodiments, the cell culture apparatus 950 may include a bottom plate 910 and one or more sidewalls 940, as shown in FIG. 9. The bottom plate 910 may define a major surface 911 and the one or more sidewalls 940 may extend from the bottom plate 910. The cell culture apparatus 950 may also include a plurality of wells 920 formed in the major surface 911 of the bottom plate 910. Each well of the plurality of wells 920 may define a microwell or a well channel (including a microwell or virtual microwell) or may include an insert that defines a microwell or a well channel (including a microwell or a virtual microwell), as described previously (e.g., with regard to FIGS. 1-8), that grows spheroids. The major surface 911 of the bottom plate 910 and the one or more sidewalls 940 define a reservoir volume. Reservoir plates described herein permit the addition of culture medium in excess of what would be typically used to fill individual shallow wells of a microwell plate and allows cells cultured in different wells to be in fluid communication.

In some embodiments, the one or more sidewalls 940 may extend farther away (e.g., a sidewall height) from the bottom plate 910 than some currently available cell culture devices, and therefore, allowing the reservoir to hold a larger than normal volume of medium. The larger capacity opportunity for the reservoir may allow an excess of culture medium to be added to the reservoir so that the spheroids may not need to rely only on the amount of medium in each individual well. In other words, the spheroids may not need to be fed with cell culture medium as frequently as spheroids growing in standard microplate wells. As shown in FIG. 9, nutrients and metabolites may be exchanged throughout the cell culture medium because the cell culture medium in the reservoir is in communication with all of the wells in the reservoir. Cell culture medium can be removed from the reservoir, if desired, to allow isolated assaying of spheroids within individual wells 920.

In various embodiments, a cell culture system can include more than one cell culture apparatus component described herein above. By way of example, the apparatus components can be stacked to form a cell culture system. Examples of stacked cell culture systems that can incorporate a cell culture apparatus component as described herein include those described in for example, (i) U.S. provisional patent application No. 62/072,015, filed on 29 Oct. 2014, entitled “MULTILAYER CULTURE VESSEL,”; (ii) U.S. provisional patent application No. 62/072,039, entitled “PERFUSION BIOREACTOR PLATFORM”, filed on 29 Oct. 2014, which provisional patent applications are each hereby incorporated herein by reference in their respective entireties to the extent that they do not conflict with the present disclosure.

The cell culture devices described herein can be used to culture cells within microwells of the apparatus in any suitable manner. For example, a method for culturing cells involves introducing cells and a cell culture medium into one or more of the plurality of microwells or well channels of a cell culture apparatus as described herein. The cell culture medium may be contained in only the microwell, or the microwell and well channel, or the well channel and hanging from the bottom aperture of the well channel. The method also involves culturing cells in the medium in the one or more plurality of microwells or virtual microwells. Culturing the cells in the one or more of the plurality of microwells may include forming a spheroid within the one or more microwells. The cells may form a spheroid due to the geometry of the cell culture medium. The geometry of the cell culture medium may be provided by the structure of the microwell or the virtual microwell that is formed as a result of the characteristics of the cell culture apparatus as described herein.

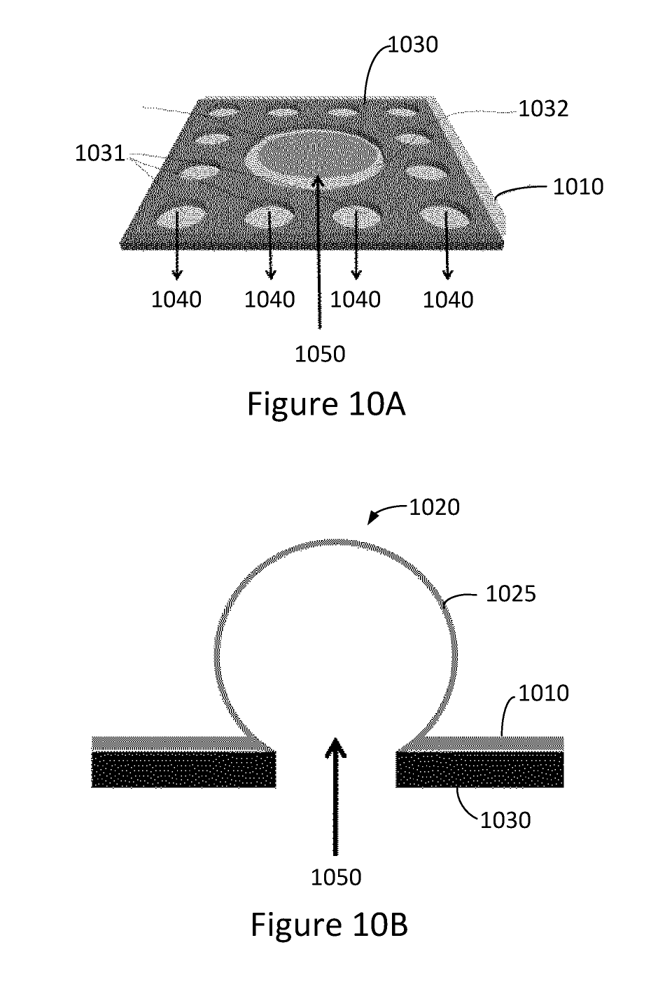

A cell culture apparatus as described herein can be manufactured in any suitable manner. In various embodiments, a method of manufacturing a cell apparatus includes retaining a substrate 1010 relative to a prefabricated support plate 1030, as shown in FIGS. 10A and 10B. The prefabricated support plate 1030 includes a first array of one or more holes 1031 and a second array of one or more holes 1032. Each hole of the second array of holes 1032 may be larger than each hole of the first array of holes 1031. Each hole of the second array of holes 1032 may be defined by a radius that may be about, e.g., greater than or equal to 20 micrometers, greater than or equal to 40 micrometers, greater than or equal to 100 micrometers, greater than or equal to 250 micrometers, greater than or equal to 500 micrometers, greater than or equal to 650 micrometers, etc. or, less than or equal to 3000 micrometers, less than or equal to 2000 micrometers, less than or equal to 1500 micrometers, less than or equal to 1000 micrometers, less than or equal to 750 micrometers, less than or equal to 400 micrometers, etc. or any range within the aforementioned values.

A vacuum 1040 may be applied to the substrate 1010 through the first array of holes 1031 to hold the substrate 1010 in contact with the prefabricated support plate 1030. The substrate 1010 may then be heated by a laser or some other suitable heating source to a temperature above a softening point of the substrate 1010. A uniform pressure 1050 of gas may then be applied (simultaneous to the heat) through the second array of holes 1032 to deform the substrate 1010 into a spherical shape 1025 at each hole of the second array of holes 1032. This deformation forms bowl shaped microbubbles that create microwells 1020 in the substrate 1010 of the cell culture apparatus. The substrate 1010 (e.g., glass, polymer material, etc.) will deform into a spherical shape 1025 due to uniform pressure distribution 1050. Duration and the magnitude of the applied pressure 1050 and the viscoelastic properties of the softened substrate 1010 or film will determine the size and shape of the blown microwell.

Regardless of how a cell culture apparatus described herein is manufactured, sidewall surfaces of each of a plurality of microwells and well channels may be formed from or coated with a cell non-adherent material.

Preferably, materials of the cell culture devices described herein that are intended to contact cells or culture media are compatible with the cells and the media. Typically, cell culture components are formed from polymeric material. Examples of suitable polymeric materials include polystyrene, polymethylmethacrylate, polyvinyl chloride, polycarbonate, polysulfone, polystyrene copolymers, fluoropolymers, polyesters, polyamides, polystyrene butadiene copolymers, fully hydrogenated styrenic polymers, polycarbonate PDMS copolymers, and polyolefins such as polyethylene, polypropylene, polymethyl pentene, polypropylene copolymers and cyclic olefin copolymers, and the like.

All scientific and technical terms used herein have meanings commonly used in the art unless otherwise specified. The definitions provided herein are to facilitate understanding of certain terms used frequently herein and are not meant to limit the scope of the present disclosure.

As used herein, singular forms “a,” “an” and “the” include plural referents unless the context clearly dictates otherwise. Thus, for example, reference to a “microwell” includes examples having two or more such “microwells” unless the context clearly indicates otherwise.

As used in this specification and the appended claims, the term “or” is generally employed in its sense including “and/or” unless the content clearly dictates otherwise. The term “and/or” means one or all of the listed elements or a combination of any two or more of the listed elements.

As used herein, “have”, “has”, “having”, “include”, “includes”, “including”, “comprise”, “comprises”, “comprising” or the like are used in their open ended inclusive sense, and generally mean “include, but not limited to”, “includes, but not limited to”, or “including, but not limited to”.

“Optional” or “optionally” means that the subsequently described event, circumstance, or component, can or cannot occur, and that the description includes instances where the event, circumstance, or component, occurs and instances where it does not.

The words “preferred” and “preferably” refer to embodiments of the disclosure that may afford certain benefits, under certain circumstances. However, other embodiments may also be preferred, under the same or other circumstances.

Furthermore, the recitation of one or more preferred embodiments does not imply that other embodiments are not useful, and is not intended to exclude other embodiments from the scope of the inventive technology.

Ranges can be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Also herein, the recitations of numerical ranges by endpoints include all numbers subsumed within that range (e.g., 1 to 5 includes 1, 1.5, 2, 2.75, 3, 3.80, 4, 5, etc.). Where a range of values is “greater than”, “less than”, etc. a particular value, that value is included within the range.

Any direction referred to herein, such as “top,” “bottom,” “left,” “right,” “upper,” “lower,” “above,” below,” and other directions and orientations are described herein for clarity in reference to the figures and are not to be limiting of an actual device or system or use of the device or system. Many of the devices, articles or systems described herein may be used in a number of directions and orientations. Directional descriptors used herein with regard to cell culture devices often refer to directions when the apparatus is oriented for purposes of culturing cells in the apparatus.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred. Any recited single or multiple feature or aspect in any one claim can be combined or permuted with any other recited feature or aspect in any other claim or claims.

It is also noted that recitations herein refer to a component being “configured” or “adapted to” function in a particular way. In this respect, such a component is “configured” or “adapted to” embody a particular property, or function in a particular manner, where such recitations are structural recitations as opposed to recitations of intended use. More specifically, the references herein to the manner in which a component is “configured” or “adapted to” denotes an existing physical condition of the component and, as such, is to be taken as a definite recitation of the structural characteristics of the component.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a cell culture apparatus comprising a structure defining a cell culture microwell include embodiments where cell culture apparatus consists of a structure defining a cell culture microwell and embodiments where a cell culture apparatus consists essentially of a structure defining a cell culture microwell.

It will be apparent to those skilled in the art that various modifications and variations can be made to the present inventive technology without departing from the spirit and scope of the disclosure. Since modifications, combinations, sub-combinations and variations of the disclosed embodiments incorporating the spirit and substance of the inventive technology may occur to persons skilled in the art, the inventive technology should be construed to include everything within the scope of the appended claims and their equivalents.

What is claimed is:

1. A cell culture device comprising:
a cell culture well, wherein the cell culture well defines a top aperture, an inner surface, and an axis extending through the top aperture,
wherein the top aperture defines a top diametric dimension measured perpendicular to the axis, and wherein the inner surface defines a first diametric dimension measured perpendicular to the axis at a widest portion of the cell culture well and a second diametric dimension measured perpendicular to the axis,
wherein the second diametric dimension is at a location along the axis farther from the top aperture than a location of the first diametric dimension along the axis,
wherein the top diametric dimension is less than the first diametric dimension, and wherein the second diametric dimension is less than the first diametric dimension.

2. A cell culture device according to claim 1, wherein the device comprises a plurality of cell culture wells.

3. A cell culture device according to claim 1, wherein the top diametric dimension is in a range from 40 to 6000 micrometers.

4. A cell culture device according to claim 1, wherein the first diametric dimension is in a range from 50 to 8000 micrometers.

5. A cell culture device according to claim 1, wherein the inner surface is non-adherent to cells.

6. A cell culture device according to claim 1, wherein a gas permeable substrate defines the inner surface.

7. A cell culture device according to claim 6, wherein the gas permeable substrate comprises a gas permeable film.

8. A cell culture device according to claim 1, wherein the device further comprises a well channel, wherein the well channel defines a top aperture, a bottom aperture, a sidewall surface extending from the top aperture to the bottom aperture, and a middle aperture between the top aperture and the bottom aperture, wherein the bottom aperture of the well channel is the top aperture of the cell culture well.

9. A cell culture device according to claim 8, wherein a diametric dimension of the top aperture of the well channel is greater than or equal to a diametric dimension of the bottom aperture of the well channel.

10. A cell culture device according to claim 8, wherein a diametric dimension of the top aperture of the well channel is greater than or equal to a diametric dimension of the middle aperture of the well channel and the diametric dimension of the middle aperture of the well channel is greater than or equal to a diametric dimension of the bottom aperture of the well channel.

11. A cell culture device according to claim 8, wherein the sidewall surface between the top and middle aperture of the well channel defines an upper sidewall surface and the sidewall surface between the middle and bottom apertures of the channel well defines a lower sidewall surface, wherein the lower sidewall surface is defined in a plane that is parallel with a plane defined by the top aperture of the well channel.

12. A cell culture device according to claim 1, further comprising a cell culturing well, wherein the device is placed within the cell culturing well.

13. A cell culture apparatus according to claim 1, wherein the cell culture microwell defines a semi-spherical shape.

14. A method of culturing cells to form a spheroid comprising:
introducing cells and a cell culture medium into a cell culture microwell of a cell culture apparatus according to claim 1; and
culturing the cells in the cell culture medium in the cell culture microwell to form a spheroid.

15. A cell culture apparatus comprising:
a structure defining a well channel, wherein the well channel defines a top aperture, a bottom aperture and a sidewall surface between the top and bottom aperture,
wherein the top aperture defines a top diametric dimension and the bottom aperture defines a bottom diametric dimension smaller than the top diametric dimension, wherein the bottom diametric dimension is in a range from 25 micrometers to 4,000 micrometers,
wherein the well channel is configured to hang a drop of cell culture fluid below the bottom aperture to form a cell culture microwell.

16. A cell culture apparatus according to claim 15, further comprising a reservoir located below the bottom aperture of the well channel, wherein the reservoir defines a top aperture, a bottom surface, and a sidewall surface extending from the top aperture to the bottom surface, wherein the sidewall surface of the reservoir is sealed to an exterior surface of the well channel that is opposite the sidewall surface of the well channel.

17. A cell culture apparatus according to claim 16, wherein bottom surface of the reservoir defines a flat planar surface that is parallel with a plane defined by the bottom aperture of the well channel.

18. A cell culture apparatus according to claim 16, wherein bottom surface of the reservoir defines parabolic shape.

19. A method of culturing cells to form a spheroid, comprising
introducing cells and a cell culture medium into a well channel of a cell culture apparatus according to claim 15 to form a hanging drop of the cell culture medium below the bottom aperture; and
culturing the cells within the hanging drop to form a spheroid.

20. A method of manufacturing a cell culture apparatus comprising:
retaining a substrate relative to a prefabricated support plate, wherein the prefabricated support plate comprises a first array of holes and a second array of holes, wherein each hole of the second array of holes is defined by a radius in a range from 20 micrometers to 3,000 micrometers;
applying a vacuum to the substrate through the first array of holes to hold the substrate in contact with the prefabricated support plate;
heating the substrate by a laser to a temperature above a softening point of the substrate; and
applying a uniform pressure of gas through the second array of holes to deform the substrate into a spherical shape at each hole of the second array of holes.
